(19) United States
(12) Patent Application Publication
(10) Pub. No.: US 2013/0309220 A1
(43) Pub. Date: Nov. 21, 2013

(54) COMPOSITIONS FOR TREATING MICROBIAL INFECTIONS

(71) Applicant: Rueben Matalon, Houston, TX (US)

(72) Inventor: Rueben Matalon, Houston, TX (US)

(21) Appl. No.: 13/872,059

(22) Filed: Apr. 26, 2013

Related U.S. Application Data
(60) Provisional application No. 61/648,776, filed on May 18, 2012.

(51) Int. Cl. A61K 38/47; A61K 31/7008

(52) U.S. Cl. CPC A61K 38/47; A61K 31/7008
USPC 424/94.61

(57) ABSTRACT

Certain embodiments are directed to methods of treating a condition associated with microbial infection in a subject having such a condition comprising administering to the subject a composition comprising an effective amount of (a) lysozyme and (b) N-acetyl glucosamine polymer.

This application claims priority to U.S. Provisional Application Ser. No. 61/648,776 filed May 18, 2012, which is incorporated herein by reference in its entirety.

### BACKGROUND OF THE INVENTION

I. Field of the Invention

Embodiments of this invention are directed generally to biology and medicine. Certain aspects are directed to anti-microbial compositions and methods of treating microbial infections.

II. Background

Necrotizing enterocolitis is a devastating illness in premature infants. The pathogenesis of NEC involves a combination of predisposing factors that leads to mucosal injury and intestinal necrosis. Intestinal ischemia, enteral feeding, bacterial colonization, and gut immaturity have all been implicated in the pathogenesis of NEC (Caplan and Jilling, 2001; Claud and Walker, 2001). NEC is rarely, if ever, observed in utero and ninety percent of infants with NEC are born preterm (Claud and Walker, 2001) making premature birth the single most common risk factor for the condition in humans. Amniotic fluid contains hormones and peptides that play a role in intestinal maturation and preparation for postnatal enteral feeding. Preterm birth may not allow for proper maturation of the gut.

A 6-10 fold increase in the incidence of NEC has been reported in formula-fed infants compared with breast-fed infants (Claud and Walker, 2001). Feeding of human milk to premature infants of less than 1500 g has been associated with poorer rates of growth and nutritional deficits (Schanler, 2001), most of their nutritional requirements are met via total parenteral nutrition administered through a central line. Furthermore, technical factors associated with the collection, storage and delivery of breast milk to premature infants renders the use of human breast milk difficult, and even unlikely in this clinical context.

Bacterial colonization has been identified as a prerequisite in the development of NEC (Caplan and Jilling, 2001). Although NEC can present in clusters (Caplan and Jilling, 2001) and displays an epidemiology reminiscent of a nosocomial infection (Boccia et al., 2001; Hentschel et al., 1999) no particular pathogen has been associated with the pathogenesis of NEC. These findings suggest that NEC may be the result of a secondary inflammatory response to the colonizing organisms rather than a direct infection. The role of bacterial colonization in the development of necrotizing enterocolitis is supported by evidence indicating that oral antibiotics reduce the incidence of NEC in low birth weight infants (Bury and Tudehope, (Cochrane Review, 2001).

Various factors have been found to have a beneficial effect against NEC, for example: breast milk (Dvorák et al., 2003; McGuire and Anthony, 2003); L-carnitine (Akisu et al., 2002), platelet-activating factor receptor antagonists (Caplan et al., 1997), intestinal Lactobacillus and Bifidobacterial supplementation (Caplan et al., 1999; Hoyos, 1999); interleukin 10 (Ozturk et al., 2002), IgA supplementation (Eibl et al., 1988), enteral antibiotic prophylaxis (Siu et al., 1998), early postnatal dexamethasone treatment (Halac et al., 1990), and neonatal formula supplemented with egg phospholipids (Carlson et al., 1998).

Despite the many agents known to have potential in the treatment of NEC in a clinical setting, the most common treatment for NEC remains a regimen of antibiotics. Systemic antibiotic therapy with ampicillin and gentamicin is typically provided unless resistant Staphylococcus epidermidis is suspected, in which case vancomycin is used instead of ampicillin. Clinamycin, metronidazole, or other anaerobic therapy is often used to treat anaerobic infections if perforation is suspected or has occurred (Neu, 1996). However, no fully preventative or therapeutic treatment for preventing or treating NEC is known. There remains a need for additional compositions and methods for treating necrotizing enterocolitis, as well as other conditions associated with microbial infections.

### SUMMARY OF THE INVENTION

Certain embodiments are directed to methods of treating a condition associated with microbial infection in a subject having such a condition comprising administering to the subject a composition comprising an effective amount of (a) lysozyme and/or (b) N-acetyl glucosamine polymer. In certain aspects the microbial infection is a gastrointestinal infection, eye infection, ear infection, nasal infection, or a throat infection. In a further aspect the condition is necrotizing enterocolitis, otitis, or respiratory infection. In still a further aspect the microbial infection is a viral infection or a bacterial infection or a fungal infection. In certain aspects the viral infection is respiratory syncytial virus (RSV) infection. In certain aspects the composition is administered in combination with one or more probiotic compositions. Probiotic compositions are generally defined as microbial dietary supplements that beneficially affect the host by improving intestinal microbial balance. The two major genera of microorganisms commonly associated with probiotics include Lactobacillus sp and Bifidobacteria sp.

In certain aspects the composition has a lysozyme concentration that is between 25, 30, 40, 50, 60, 70, 80, 90, 100, 200 mg/L to 100, 200, 300, 400, 500, 600, 700, 800 mg/L, including all values and ranges there between. In certain aspects the lysozyme is present in a concentration of between 100 mg/L to 800 mg/L. In certain aspects lysozyme is a recombinant lysozyme. In a further aspect the lysozyme is a bacterial, fungal, or animal lysozyme. In still further aspects the lysozyme is mammalian lysozyme, for example human lysozyme.

In certain aspects the N-acetyl glucosamine polymer concentration is between 1, 5, 10, 15, 20, 25, 30 g/L to 25, 30, 40, 45, 50 g/L, including all values and ranges there between. In a further aspect the N-acetyl glucosamine polymer is at a concentration of 15 to 25 g/L. In certain aspects the N-acetyl glucosamine polymer comprises at least 4, 5, 6, 7, 8, 9, 10 or more N-acetyl glucosamine units. In certain aspects the N-acetyl glucosamine monomer is 2-(acetylamino)-2-deoxy-D-glucose. The monomers can be either deacetylated or acetylated or a mixture of both. In certain aspects the N-acetyl glucosamine polymer is chitin and/or chitosan.

In certain aspects the subject is a newborn or an infant. The term “newborn” refers to a mammal, such as a human, that is less than 5 days old. The term “infant” refers to a mammal, such as human, that is less than or equal to 2 years old. In further aspects the newborn is a premature newborn. A premature newborn is a mammal, such as a human, that is less than 5 days old and was born before the 37^{th} week of gestational age. The term “gestational age” means fetal age of a newborn, calculated from the number of completed weeks since the first day of the mother's last menstrual period to the date of birth.

Probiotic composition can comprise one or more of a Lactobacillus acidophilus, Lactobacillus amylovorus, Lactobacillus brevis, Lactobacillus casei, Lactobacillus delbrueckii, Lactobacillus fermentum, Lactobacillus helvaticus, Lactobacillus paracasei, Lactobacillus pentosus, Lactobacillus plantarum, Lactobacillus reuteri, Bacillus coagulans, Bifidobacterium animalis, Bifidobacterium longum, Lactobacillus johnsonii, Lactobacillus rhamnosus, and/or Bifidobacterium bifidum.

In certain aspects administration of the composition is by enteric administration, nasal administration, inhalation, inspiration, or topical administration. Topical administration includes, but is not limited to administration by ear drop or eye drop. Enteric administration includes, but is not limited to oral administration. In certain, aspect the composition is formulated for inhalation or delivery via a nebulizer.

Certain embodiments are directed to an infant food supplement. The infant food supplement comprises between 5 g/L to 50 g/L of N-acetyl glucosamine polymer or a mixture thereof, and between 25 mg/L to 800 mg/L of lysozyme.

Further embodiments include an anti-microbial ear drop composition comprising between 5 g/L to 50 g/L of N-acetyl glucosamine polymer or a mixture thereof, and between 25 mg/L to 800 mg/L of lysozyme.

Other embodiments of the invention are discussed throughout this application. Any embodiment discussed with respect to one aspect of the invention applies to other aspects of the invention as well and vice versa.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION OF THE INVENTION

Certain embodiments are directed to methods of treating a condition associated with microbial infection, for example a viral or bacterial infection, in a subject having such a condition comprising administering to the subject a composition comprising an effective amount of (a) lysozyme and/or (b) N-acetyl glucosamine polymer. In certain aspects the microbial infection is a respiratory, gastrointestinal infection, eye infection, ear infection, nasal infection, or a throat infection. In a further aspect the condition is lung infection, necrotizing enterocolitis or otitis. In certain aspects the microbial infection is a viral infection.

Chitin and Chitosan are polymers of N-acetyl glucosamine. Chitin, chitosan and their oligosaccharides can induce the production of the enzyme lysozyme (Zhang et al., 1993). Human breast milk is known to contain lysozyme. Lysozyme has anti-bacterial activities and was first discovered by Alexander Fleming as the first antibiotic before the discovery of penicillin in 1922. Lysozyme is found in tears, saliva, GI tract and serum.

Chitin, Chitosan, and their polymers are substrates and potent inducers of Lysozyme in the GI tract and other tissues. The methods described herein use Chitin, Chitosan and their oligosaccharides in combination with or without Lysozyme to serve as prebiotic or anti-microbial. In certain aspects the compositions described can be used as a supplement to infant formula.

Chitin, Chitosan and their polymers together with Lysozyme can be used to treat respiratory infections; and inflammatory bowel diseases such as ulcerative colitis, Crohn's disease, and the like. In certain aspects, chitin, chitosan or their polymers, and Lysozyme can be given by enema.

Chitin ((C₈H₁₃O₅N)n) is a long-chain polymer of a N-acetylglucosamine, a derivative of glucose. Chitin is the main component of the cell walls of fungi, the exoskeletons of arthropods such as crustaceans (e.g., crabs, lobsters and shrimps) and insects, the radulas of mollusks, and the beaks of cephalopods, including squid and octopuses. In terms of structure, chitin may be compared to the polysaccharide cellulose and, in terms of function, to the protein keratin.

Chitosan is a linear polysaccharide composed of randomly distributed β-(1-4)-linked D-glucosamine (deacetylated unit) and N-acetyl-D-glucosamine (acetylated unit). Chitosan is produced commercially by deacetylation of chitin. The degree of deacetylation (% DD) can be determined by NMR spectroscopy, and the % DD in commercial chitosans is in the range 60-100%. On average, the molecular weight of commercially produced chitosan is between 3800 to 20,000 daltons. A common method for the synthesis of chitosan is the deacetylation of chitin using sodium hydroxide in excess as a reagent and water as a solvent.

The amino group in chitosan has a pKa value of ˜6.5, which leads to a protonation in acidic to neutral solution with a charge density dependent on pH and the % DA-value. This makes chitosan water soluble and a bioadhesive which readily binds to negatively charged surfaces such as mucosal membranes. Chitosan enhances the transport of polar drugs across epithelial surfaces, and is biocompatible and biodegradable.

Lysozyme, also known as muramidase or N-acetylmuramide glycanhydrolase, are glycoside hydrolases, enzymes (EC 3.2.1.17) that damage bacterial cell walls by catalyzing hydrolysis of 1,4-beta-linkages between N-acetylmuramic acid and N-acetyl-D-glucosamine residues in a peptidoglycan and between N-acetyl-D-glucosamine residues in chitodextrins. Lysozyme is abundant in a number of secretions, such as tears, saliva, human milk, and mucus. It is also present in cytoplasmic granules of the polymorphonuclear neutrophils (PMN). In humans, the lysozyme enzyme is encoded by the LYZ gene.

Lysozyme is part of the innate immune system. Children fed infant formula lacking lysozyme in their diet have three times the rate of diarrheal disease. Since lysozyme is a natural form of protection from pathogens like Salmonella, E. coli, and Pseudomonas, a deficiency due to infant formula feeding can lead to increased incidence of disease. Whereas the skin is a protective barrier due to its dryness and acidity, the conjunctiva (membrane covering the eye) is, instead, protected by secreted enzymes, mainly lysozyme and defensin.

### I. Innate Immune System.

The adaptive immune system may take days or weeks after an initial infection to have an effect. However, most organisms are under constant assault from pathogens that must be kept in check by the faster-acting innate immune system. Innate immunity defends against pathogens by rapid responses coordinated through “innate” mechanisms that recognize a wide spectrum of conserved pathogenic components. Most studies of innate immunity have focused on leukocytes such as neutrophils, macrophages, and natural killer cells. However, epithelial cells play key roles in innate defenses that include providing a mechanical barrier to microbial entry, signaling to leukocytes, and directly killing pathogens. Importantly, all these defenses are highly inducible in response to the sensing of microbial and host products.

The “first-line” defense includes physical and chemical barriers to infection, such as skin and mucus coating of the gut and airways, physically preventing the interaction between the host and the pathogen. Pathogens, which penetrate these barriers, encounter constitutively-expressed anti-microbial molecules (e.g., lysozyme) that restrict the infection. The “second-line” defense includes phagocytic cells (macrophages and neutrophil granulocytes) that can engulf (phagocytose) foreign substances.

Phagocytosis involves chemotaxis, where phagocytic cells are attracted to microorganisms by means of chemotactic chemicals such as microbial products, complement, damaged cells and white blood cell fragments. Chemotaxis is followed by adhesion, where the phagocyte sticks to the microorganism. Adhesion is enhanced by opsonization, where proteins like opsonins are coated on the surface of the microbe. This is followed by ingestion, in which the phagocyte extends projections, forming pseudopods that engulf the foreign organism. Finally, the pathogen is digested by the enzymes in the lysosome, involving reactive oxygen species and proteases.

In addition, anti-microbial proteins may be activated if a pathogen passes through a physical barrier. There are several classes of antimicrobial proteins, such as acute phase proteins e.g., C-reactive protein, which enhances phagocytosis and activates complement when it binds the C-protein of S. pneumonia; lysozyme; and the complement system.

The complement system is a very complex group of serum proteins, which is activated in a cascade fashion. Three different pathways are involved in complement activation: (a) a classical pathway that recognizes antigen-antibody complexes, (b) an alternative pathway that spontaneously activates on contact with pathogenic cell surfaces, and (c) a mannose-binding lectin pathway that recognizes mannose sugars, which tend to appear only on pathogenic cell surfaces. A cascade of protein activity follows complement activation; this cascade can result in a variety of effects, including opsonization of the pathogen, destruction of the pathogen by the formation and activation of the membrane attack complex, and inflammation.

Interferons are also anti-microbial proteins. These molecules are proteins that are secreted by virus-infected cells. These proteins then diffuse rapidly to neighboring cells, inducing the cells to inhibit the spread of the viral infection. Essentially, these anti-microbial proteins act to prevent the cell-to-cell proliferation of viruses.

### II. Microbial Associated Conditions

Necrotizing enterocolitis (NEC) is a medical condition primarily seen in premature infants, where portions of the bowel undergo necrosis (tissue death). The condition is typically seen in premature infants, and the timing of its onset is generally inversely proportional to the gestational age of the baby at birth, i.e. the earlier a baby is born, the later signs of NEC are typically seen. Initial symptoms include feeding intolerance, increased gastric residuals, abdominal distension and bloody stools. Symptoms may progress rapidly to abdominal discoloration with intestinal perforation and peritonitis and systemic hypotension requiring intensive medical support.

NEC has no definitive known cause. An infectious agent has been suspected, as cluster outbreaks in neonatal intensive care units (NICUs) have been seen, but no common organism has been identified. Pseudomonas aeruginosa is suspected for causing necrotising enterocolitis in premature infants and neutropaenic cancer patients, often secondary to gut colonization. A combination of intestinal flora, inherent weakness in the neonatal immune system, empirical antibiotic use for 5 days or more, alterations in mesenteric blood flow and milk feeding may be factors. The most common area of the bowel affected by NEC is near the ileocecal valve (the site of transition between the small and large bowel). NEC is almost never seen in infants before oral feedings are initiated. It is estimated that formula feeding increases the risk of NEC by tenfold compared to infants who are fed breastmilk alone. Expressed breast milk protects the premature infant not only by its antiinfective effect and its immunoglobulin agents but also from its rapid digestion.

Treatment consists primarily of supportive care including providing bowel rest by stopping enteral feeds, gastric decompression with intermittent suction, fluid repletion to correct electrolyte abnormalities and third space losses, support for blood pressure, parenteral nutrition, and prompt antibiotic therapy. Monitoring is clinical, although serial supine and left lateral decubitus abdominal roentgenograms should be performed every 6 hours. Where the disease is not halted through medical treatment alone, or when the bowel perforates, immediate emergency surgery to resect the dead bowel is generally required, although abdominal drains may be placed in very unstable infants as a temporizing measure. Surgery may require a colostomy, which may be able to be reversed at a later time. Some children may suffer later as a result of short bowel syndrome if extensive portions of the bowel had to be removed.

Typical recovery from NEC if medical, non-surgical treatment succeeds, includes 10-14 days or more without oral intake and then demonstrated ability to resume feedings and gain weight. Recovery from NEC alone may be compromised by co-morbid conditions that frequently accompany prematurity. Longterm complications of medical NEC include bowel obstruction and anemia. Despite a significant mortality risk, long-term prognosis for infants undergoing NEC surgery is improving, with survival rates of 70-80%. “Surgical NEC” survivors are at-risk for complications including short bowel syndrome, and neurodevelopmental disability.

Respiratory infections, particularly upper respiratory infections (“URIs”) are very common and cause substantial suffering and hundreds of millions of dollars of economic loss every year. The majority of the pathogens contributing to upper respiratory tract infections are spread through air and through direct contact by touching of hands to infected surfaces and then touching hands to eyes, nose, or mouth. The nasopharynx, nasal passages, and sinus cavities all play an important role in filtering and housing the majority of these pathogens.

Upper respiratory infections (“URIs”) such as Respiratory Syncytial Virus (RSV) infection, the common cold, and influenza (the “flu”) are generally preceded by one or more of a number of minor symptoms such as minor headaches, minor eye aches, minor ear aches, minor sore throat, minor body ache, minor nasal congestion, slight runny nose, minor cough, slight itching or scratchiness in the throat, itchiness in the ear, minor hoarseness, roughness in the eyes when moving the eyes or when blinking, sneezing, minor chills or shivers at normal room temperature, feeling abnormally warm at normal room temperature. These symptoms are characterized here as “minor” in the sense that they are just barely detectable and felt by the affected individual, they have typically just begun to be felt within the last hour or so, and they do not yet interfere significantly with normal daily activities. People generally ignore these symptoms, hoping they will just go away, or thinking that they may be caused by a minor allergy or irritation from dust or similar external cause. They wait for symptoms to develop into something more serious before beginning treatment. These symptoms can be called “major symptoms.” Examples include sore throat, fever, muscle aches, serious headache, etc. These symptoms are characterized here as “major” in that they cause significant discomfort to the affected individual, persist for an extended period of time (hours to days or even weeks), cause a general feeling of illness, and interfere with normal daily activities including work, play, and sleep.

Treatment options for URIs are very limited. A variety of over-the-counter remedies are available, most of which have limited efficacy, and most URIs are basically allowed to run their course until the body's defense mechanisms eventually succeed in fighting off the infection. The present invention is directed to providing new compositions for treatment before or during a URI.

### III. Formulations and Administration

The pharmaceutical compositions disclosed herein may be administered via the alimentary system of a subject or by topical administration. In certain aspects the compositions are administered by oral administration or by ear or eye drops. Compositions may be prepared in water. Dispersions may be prepared in glycerol, liquid polyethylene glycols and mixtures thereof, and in oils. Under ordinary conditions of storage and use, these preparations contain a preservative to prevent the growth of microorganisms. The pharmaceutical forms suitable for oral administration include sterile aqueous solutions or dispersions, and sterile powders for the extemporaneous preparation of orally delivered or topically applied solutions or dispersions. In all cases the form is typically sterile and capable of ingestion or application directly or through some intermediary process or device. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like.

Some variation in dosage will necessarily occur depending on the condition of the subject being treated and the particular circumstances. The person responsible for administration will, in any event, determine the appropriate dose for the individual subject. Moreover, for human administration, preparations should meet sterility, pyrogenicity, general safety, and purity standards as required by FDA Office of Biologics standards or other similar organizations.

Sterile compositions are prepared by incorporating the active components in the required amount in the appropriate solvent with various other ingredients enumerated above, as required, followed by, for example, filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some methods of preparation are vacuum-drying and freeze-drying techniques which yield a powder of the active ingredient plus any additional desired ingredient from a previously sterile-filtered solution.

According to the invention, at least one pharmaceutical composition can be delivered by any of a variety of inhalation or nasal devices known in the art for administration of a therapeutic agent by inhalation or nasal instillation. Other devices suitable for directing administration are also known in the art.

A spray comprising a pharmaceutical composition of the present invention can be produced by forcing a suspension or solution of a composition through a nozzle under pressure. The nozzle size and configuration, the applied pressure, and the liquid feed rate can be chosen to achieve the desired output and particle size.

Pulmonary/respiratory drug delivery can be implemented by different approaches, including liquid nebulizers, aerosol-based metered dose inhalers (MDI's), sprayers, dry powder dispersion devices and the like. Such methods and compositions are well known to those of skill in the art, as indicated by U.S. Pat. Nos. 6,797,258, 6,794,357, 6,737,045, and 6,488,953, all of which are incorporated by reference. According to the invention, at least one pharmaceutical composition can be delivered by any of a variety of inhalation or nasal devices known in the art for administration of a therapeutic agent by inhalation. Other devices suitable for directing pulmonary or nasal administration are also known in the art. Typically, for pulmonary administration, at least one pharmaceutical composition is delivered in a particle size effective for reaching the lower airways of the lung or sinuses. Some specific examples of commercially available inhalation devices suitable for the practice of this invention are Turbohaler™ (Astra), Rotahaler® (Glaxo), Diskus® (Glaxo), Spiros™ inhaler (Dura), devices marketed by Inhale Therapeutics, AERx™ (Aradigm), the Ultravent® nebulizer (Mallinckrodt), the Acorn II® nebulizer (Marquest Medical Products), the Ventolin® metered dose inhaler (Glaxo), the Spinhaler® powder inhaler (Fisons), or the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a subject. The preparation of an aqueous composition that contains a polypeptide or peptide as an active ingredient is well understood in the art.

A. Infant Formula

Ready-to-feed infant formulas can be prepared by the appropriate blending of ingredients, including varying proportions of animal fats to yield a composition having a protein, mineral, carbohydrate, and fatty acid composition approximating that of human milk fat. The mixture can be incorporated into an infant formula in concentrated powder or liquid form, or in ready-to-use form. In certain aspects the infant formula can be supplemented with effective amount of (a) lysozyme and (b) N-acetyl glucosamine polymer. In certain aspects the composition has a lysozyme concentration that is between 25, 30, 40, 50, 60, 70, 80, 90, 100, 200 mg/L to 100, 200, 300, 400, 500, 600, 700, 800 mg/L, including all values and ranges there between. In certain aspects the lysozyme is present in a concentration of between 100 mg/L to 800 mg/L. In certain aspects the N-acetyl glucosamine polymer concentration is between 1, 5, 10, 15, 20, 25, 30 g/L to 25, 30, 40, 45, 50 g/L, including all values and ranges there between. In a further aspect the N-acetyl glucosamine polymer is at a concentration of 15 to 25 g/L. In certain aspects the N-acetyl glucosamine polymer comprises at least 4, 5, 6, 7, 8, 9, 10 or more N-acetyl glucosamine units. In certain aspects the N-acetyl glucosamine monomer is 2-(acetylamino)-2-deoxy-D-glucose. The monomers can be either deacetylated or acetylated or a mixture of both.

The infant formula can comprise a source of carbohydrate such as sucrose or dextrose, non-fat milk, water, and edible fats. The edible fats may be any food-grade fat including, but not limited to, coconut oil, oleo oil, peanut oil, butterfat, soybean oil, olive oil, babassu oil, mutton tallow, and the like. Among the fatty acids that are provided by these fats are lauric acid, myristic acid, palmitic acid, stearic acid, oleic acid, and linoleic acid.

A typical ready-to-feed formulation for infants comprises, when diluted to feeding concentrations, from about 1-5% by weight fat, from about 0.01 to about 0.5% by weight immunoglobulins as appropriate, from about 4-10% by weight carbohydrate in a quantity substantially to mimic the carbohydrate content of human mother's milk, from about 0.5 to 4% by weight protein in a quantity substantially to mimic the protein content of human mother's milk, optional vitamins and minerals as required, a total solids content of from about 8 to 17% by weight, and the remainder water.

A non-limiting protein source for use in infant formula is electrodialyzed whey or electrodialyzed skim milk or milk whey, although other protein sources are also available and may be used. Sugars include food grade substances such as glucose, dextrose, sucrose, or edible lactose. The following vitamins and minerals may also be incorporated in the infant formula: calcium, phosphorus, potassium, sodium, chloride, magnesium, manganese, iron, copper, zinc, selenium, iodine, and vitamins A, E, D, and B complex. These micronutrients are added in the form of commonly accepted nutritional compounds in amounts equivalent to those present in human milk on a per calories basis.

To prepare an infant formula the fat soluble vitamins are dissolved in the mixture of fatty acids and the remaining formula ingredients are dissolved in the water. The fat mixture with the dissolved vitamins and the water solution are then mixed and homogenized. Adequate amounts of other trace minerals are present in the electrodialyzed whey and non-fat milk.

The infant formula can be sterilized and subsequently used on a ready-to-feed basis, or can be stored as a concentrate. The concentrate can be prepared by spray drying the formula, and the formula can be reconstituted by rehydrating the concentrate. The infant formula can be a stable liquid or powder, and has a suitable shelf life.

B. Combination Treatments

The compositions and methods of the present invention may be used in the context of a number of therapeutic or prophylactic applications. In order to increase the effectiveness of a treatment with the compositions of the present invention or to augment the protection of another therapy (second therapy), e.g., anti-microbial therapy, it may be desirable to combine these compositions and methods with other agents and methods effective in the treatment, reduction of risk of infection, or prevention of diseases and pathologic conditions, for example, anti-bacterial, anti-viral, and/or anti-fungal treatments.

Various combinations may be employed; for example, a composition comprising lysozyme and a N-acetylglucoamin polymer is “A” and the secondary therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of a composition of the present invention to a subject will follow general protocols for the administration via the alimentary system or topical administration, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

1. Anti-Virals

In certain aspects of the invention an anti-viral agent may be used in combination with a compositions. Antiviral drugs are a class of medication used specifically for treating viral infections and they should be distinguished from viricides, which actively deactivate virus particles outside the body. Most of the antivirals now available are designed to help deal with HIV, herpes viruses, the hepatitis B and C viruses, and influenza A and B viruses. Anti-viral agents useful in the invention include, but are not limited to, immunoglobulins, amantadine, interferons, nucleotide analogues, and protease inhibitors.

One anti-viral strategy is to interfere with the ability of a virus to infiltrate a target cell. This stage of viral replication can be inhibited by using agents which mimic the virus-associated protein (VAP) and bind to the cellular receptors. Or by using agents which mimic the cellular receptor and bind to the VAP. This includes anti-VAP antibodies, receptor anti-idiotypic antibodies, extraneous receptor and synthetic receptor mimics. Two such “entry-blockers,” amantadine and rimantadine, have been introduced to combat influenza.

A second approach to anti-viral therapy is to target the processes that synthesize virus components after a virus invades a cell. One way of doing this is to develop nucleotide or nucleoside analogues that look like the building blocks of RNA or DNA, but deactivate the enzymes that synthesize the RNA or DNA once the analog is incorporated. Nucleotide analogs include, but are not limited to ribivirin, vidarabine, acyclovir, gangcyclovir, zidovudine, didanosine, zalcitabine, stavudine, and lamivudine.

Yet another antiviral technique is a set of drugs based on ribozymes, which are enzymes that will cut apart viral RNA or DNA at selected sites. In their natural course, ribozymes are used as part of the viral manufacturing sequence, but these synthetic ribozymes are designed to cut RNA and DNA at sites that will disable them.

Some viruses include an enzyme known as a protease that cuts viral protein chains apart so they can be assembled into their final configuration. HIV includes a protease, and so considerable research has been performed to find “protease inhibitors” to attack HIV at that phase of its life cycle. Protease inhibitors became available in the 1990s and have proven effective, though they can have unusual side effects, for example causing fat to build up in unusual places. Improved protease inhibitors are now in development.

The final stage in the life cycle of a virus is the release of completed viruses from the host cell, and this step has also been targeted by antiviral drug developers. Two drugs named zanamivir (Relenza™) and oseltamivir (Tamiflu™) that have been introduced to treat influenza prevent the release of viral particles by blocking a molecule named neuraminidase that is found on the surface of flu viruses, and also seems to be constant across a wide range of flu strains.

Anti-viral agents include, but are not limited to abacavir; acemannan; acyclovir; acyclovir sodium; adefovir; alovudine; alvircept sudotox; amantadine hydrochloride; amprenavir; aranotin; arildone; atevirdine mesylate; avridine; cidofovir; cipamfylline; cytarabine hydrochloride; delavirdine mesylate; desciclovir; didanosine; disoxaril; edoxudine; efavirenz; enviradene; enviroxime; famciclovir; famotine hydrochloride; fiacitabine; fialuridine; fosarilate; trisodium phosphonoformate; fosfonet sodium; ganciclovir; ganciclovir sodium; idoxuridine; indinavir; kethoxal; lamivudine; lobucavir; memotine hydrochloride; methisazone; nelfinavir; nevirapine; penciclovir; pirodavir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin.

In certain embodiments an anti-viral is ribivirin and high dose ribivirin. Ribavirin is an anti-viral drug that is active against a number of DNA and RNA viruses. It is a member of the nucleoside antimetabolite drugs that interfere with duplication of viral genetic material. Though not effective against all viruses, ribavirin has wide range of activity, including important activities against influenzas, flaviviruses, and agents of many viral hemorrhagic fevers.

Typically, the oral form of ribavirin is used in the treatment of hepatitis C, in combination with pegylated interferon drugs. The aerosol form has been used in the past to treat respiratory syncytial virus-related diseases in children. However, its efficacy has been called into question by multiple studies, and most institutions no longer use it.

2. Anti-Bacterials

Examples of anti-bacterials include, but are not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines. Anti-bacterials also include, but are not limited to: Acedapsone, Acetosulfone Sodium, Alamecin, Alexidine, Amdinocillin, Amdinocillin Pivoxil, Amicycline, Amifloxacin, Amifloxacin Mesylate, Amikacin, Amikacin Sulfate, Aminosalicylic acid, Aminosalicylate sodium, Amoxicillin, Amphomycin, Ampicillin, Ampicillin Sodium, Apalcillin Sodium, Apramycin, Aspartocin, Astromicin Sulfate, Avilamycin, Avoparcin, Azithromycin, Azlocillin, Azlocillin Sodium, Bacampicillin Hydrochloride, Bacitracin, Bacitracin Methylene Disalicylate, Bacitracin Zinc, Bambermycins, Benzoylpas Calcium, Berythromycin, Betamicin Sulfate, Biapenem, Biniramycin, Biphenamine Hydrochloride, Bispyrithione Magsulfex, Butikacin, Butirosin Sulfate, Capreomycin Sulfate, Carbadox, Carbenicillin Disodium, Carbenicillin Indanyl Sodium, Carbenicillin Phenyl Sodium, Carbenicillin Potassium, Carumonam Sodium, Cefaclor, Cefadroxil, Cefamandole, Cefamandole Nafate, Cefamandole Sodium, Cefaparole, Cefatrizine, Cefazaflur Sodium, Cefazolin, Cefazolin Sodium, Cefbuperazone, Cefdinir, Cefepime, Cefepime Hydrochloride, Cefetecol, Cefixime, Cefinenoxime Hydrochloride, Cefinetazole, Cefinetazole Sodium, Cefonicid Monosodium, Cefonicid Sodium, Cefoperazone Sodium, Ceforanide, Cefotaxime Sodium, Cefotetan, Cefotetan Disodium, Cefotiam Hydrochloride, Cefoxitin, Cefoxitin Sodium, Cefpimizole, Cefpimizole Sodium, Cefpiramide, Cefpiramide Sodium, Cefpirome Sulfate, Cefpodoxime Proxetil, Cefprozil, Cefroxadine, Cefsulodin Sodium, Ceftazidime, Ceftibuten, Ceftizoxime Sodium, Ceftriaxone Sodium, Cefuroxime, Cefuroxime Axetil, Cefuroxime Pivoxetil, Cefuroxime Sodium, Cephacetrile Sodium, Cephalexin, Cephalexii Hydrochloride, Cephaloglycini, Cephaloridine, Cephalothin Sodium, Cephapirin Sodium, Cephradine, Cetocycline Hydrochloride, Cetophenicol, Chloramphenicol, Cliloramphenicol Palmitate, Chloramphenicol Pantotheniate Complex, Chloramphenicol Sodium Succinate, Chlorhexidine Phosphanilate, Chloroxylenol, Chlortetracycline Bisulfate, Chlortetracycline Hydrochloride, Cinoxacin, Ciprofloxacin, Ciprofloxacin Hydrochloride, Cirolemycin, Clarithromycin, Clinafloxacin Hydrochloride, Clildamycin, Clindamycin Hydrochloride, Clindamycin Palmitate Hydrochloride, Clindamycin Phosphate, Clofazimine, Cloxacillin Benzathine, Cloxacillin Sodium, Cloxyquin, Colistimethate Sodium, Colistin Sulfate, Coumermycin, Coumermycin Sodium, Cyclacillin, Cycloserine, Dalfopristin, Dapsone, Daptomycin, Demeclocycine, Demeclocycine Hydrochloride, Demecycline, Denofungin, Diaveridine, Dicloxacillin, Dicloxacillin Sodium, Dihydrostreptomycin Sulfate, Dipyrithione, Dirithromycin, Doxycycline, Doxycycline Calcium, Doxycycline Fosfatex, Doxycycline Hyclate, Droxacin Sodium, Enoxacin, Epicillin, Epitetracycline Hydrochloride, Erythromycin, Erythromycin Acistrate, Erythromycin Estolate, Erythromycin Ethylsuccinate, Erythromycin Gluceptate, Erythromycin Lactobionate, Erythromycin Propionate, Erythromycin Stearate, Ethambutol Hydrochloride, Ethionamide, Fleroxacin, Floxacillin, Fludalanine, Flumequine, Fosfomycin, Fosfomycin Tromethamine, Fumoxicillin, Furazolium Chloride, Furazolium Tartrate, Fusidate Sodium, Fusidic Acid, Gentamicin Sulfate, Gloximonam, Gramicidin, Haloprogin, Hetacillin, Hetacillin Potassium, Hexedine, Ibafloxacin, Imipenem, Isoconazole, Isepamicin, Isoniazid, Josamycin, Kanamycin Sulfate, Kitasamycin, Levofuraltadone, Levopropylcillin Potassium, Lexithromycin, Lincomycin, Lincomycin Hydrochloride, Lomefloxacin, Lomefloxacin Hydrochloride, Lomefloxacin Mesylate, Loracarbef, Mafenide, Meclocycline, Meclocycline Sulfosalicylate, Megalomicin Potassium Phosphate, Mequidox, Meropenem, Methacycline, Methacycline Hydrochloride, Methenamine, Methenamine Hippurate, Methenamine Mandelate, Methicillin Sodium, Metioprim, Metronidazole Hydrochloride, Metronidazole Phosphate, Mezlocillin, Mezlocillin Sodium, Minocycline, Minocycline Hydrochloride, Mirincamycin Hydrochloride, Monensin, Monensin Sodium, Nafcillin Sodium, Nalidixate Sodium, Nalidixic Acid, Natamycin, Nebramycin, Neomycin Palmitate, Neomycin Sulfate, Neomycin Undecylenate, Netilmicin Sulfate, Neutramycin, Nifuradene, Nifuraldezone, Nifuratel, Nifuratrone, Nifurdazil, Nifurimide, Nifuirpirinol, Nifurquinazol, Nifurthiazole, Nitrocycline, Nitrofurantoin, Nitromide, Norfloxacin, Novobiocin Sodium, Ofloxacin, Ormetoprim, Oxacillin Sodium, Oximonam, Oximonam Sodium, Oxolinic Acid, Oxytetracycline, Oxytetracycline Calcium, Oxytetracycline Hydrochloride, Paldimycin, Parachlorophenol, Paulomycin, Pefloxacin, Pefloxacin Mesylate, Penamecillin, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentizidone Sodium, Phenyl Aminosalicylate, Piperacillin Sodium, Pirbenicillin Sodium, Piridicillin Sodium, Pirlimycin Hydrochloride, Pivampicillin Hydrochloride, Pivampicillin Pamoate, Pivampicillin Probenate, Polymyxin B Sulfate, Porfiromycin, Propikacin, Pyrazinamide, Pyrithione Zinc, Quindecamine Acetate, Quinupristin, Racephenicol, Ramoplanin, Ranimycin, Relomycin, Repromicin, Rifabutin, Rifametane, Rifamexil, Rifamide, Rifampin, Rifapentine, Rifaximin, Rolitetracycline, Rolitetracycline Nitrate, Rosaramicin, Rosaramicin Butyrate, Rosaramicin Propionate, Rosaramicin Sodium Phosphate, Rosaramicin Stearate, Rosoxacin, Roxarsone, Roxithromycin, Sancycline, Sanfetrinem Sodium, Sarmoxicillin, Sarpicillin, Scopafungin, Sisomicin, Sisomicin Sulfate, Sparfloxacin, Spectinomycin Hydrochloride, Spiramycin, Stallimycin Hydrochloride, Steffimycin, Streptomycin Sulfate, Streptonicozid, Sulfabenz, Sulfabenzamide, Sulfacetamide, Sulfacetamide Sodium, Sulfacytine, Sulfadiazine, Sulfadiazine Sodium, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, Sulfasalazine, Sulfasomizole, Sulfathiazole, Sulfazamet, Sulfisoxazole, Sulfisoxazole Acetyl, Sulfisoxazole Diolamine, Sulfomyxin, Sulopenem, Sultamicillin, Suncillin Sodium, Talampicillin Hydrochloride, Teicoplanin, Temafloxacin Hydrochloride, Temocillin, Tetracycline, Tetracycline Hydrochloride, Tetracycline Phosphate Complex, Tetroxoprim, Thiamphenicol, Thiphencillin Potassium, Ticarcillin Cresyl Sodium, Ticarcillin Disodium, Ticarcillin Monosodium, Ticlatone, Tiodonium Chloride, Tobramycin, Tobramycin Sulfate, Tosufloxacin, Trimethoprim, Trimethoprim Sulfate, Trisulfapyrimidines, Troleandomycin, Trospectomycin Sulfate, Tyrothricin, Vancomycin, Vancomycin Hydrochloride, Virginiamycin, and/or Zorbamycin.

3. Anti-Fungals

Anti-fungal agents include, but are not limited to, azoles, imidazoles, polyenes, posaconazole, fluconazole, itraconazole, amphotericin B, 5-fluorocytosine, miconazole, ketoconazole, Myambutol (Ethambutol Hydrochloride), Dapsone (4,4′-diaminodiphenylsulfone), Paser Granules (aminosalicylic acid granules), rifapentine, Pyrazinamide, Isoniazid, Rifadin IV, Rifampin, Pyrazinamide, Streptomycin Sulfate and Trecator-SC (Ethionamide) and/or voriconazole (Vfend™).

4. Other Agents

In certain aspects of the invention an anti-inflammatory agent may be used in combination with a StIR composition.

Steroidal anti-inflammatories for use herein include, but are not limited to fluticasone, beclomethasone, any pharmaceutically acceptable derivative thereof, and any combination thereof. As used herein, a pharmaceutically acceptable derivative includes any salt, ester, enol ether, enol ester, acid, base, solvate or hydrate thereof. Such derivatives may be prepared by those of skill in the art using known methods for such derivatization.

Fluticasone—Fluticasone propionate is a synthetic corticosteroid and has the empirical formula C₂₅H₃₁F₃O₅S. It has the chemical name S-(fluromethyl)6α,9-difluoro-11β-17-dihydroxy-16α-methyl-3-oxoandrosta-1,4-diene-17β-carbothioate,17-propionate. Fluticasone propionate is a white to off-white powder with a molecular weight of 500.6 and is practically insoluble in water, freely soluble in dimethyl sulfoxide and dimethylformamide, and slightly soluble in methanol and 95% ethanol.

In an embodiment, the formulations of the present invention may comprise a steroidal anti-inflammatory (e.g., fluticasone propionate)

Beclomethasone—In certain aspects the steroidal anti-inflammatory can be beclomethasone dipropionate or its monohydrate. Beclomethasone dipropionate has the chemical name 9-chloro-11b,17,21-trihydroxy-16b-methylpregna-1,4-diene-3,20-doine17,21-dipropionate. The compound may be a white powder with a molecular weight of 521.25; and is very slightly soluble in water (Physicians' Desk Reference), very soluble in chloroform, and freely soluble in acetone and in alcohol.

Providing steroidal anti-inflammatories according to the present invention may enhance the compositions and methods of the invention by, for example, attenuating any unwanted inflammation. Examples of other steroidal anti-inflammatories for use herein include, but are not limited to, betamethasone, triamcinolone, dexamethasone, prednisone, mometasone, flunisolide and budesonide.

In accordance with yet another aspect of the invention, the non-steroidal anti-inflammatory agent may include aspirin, sodium salicylate, acetaminophen, phenacetin, ibuprofen, ketoprofen, indomethacin, flurbiprofen, diclofenac, naproxen, piroxicam, tebufelone, etodolac, nabumetone, tenidap, alcofenac, antipyrine, amimopyrine, dipyrone, animopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, meclofenamic acid, mefenamic acid, niflumic acid, salidifamides, sulindac, suprofen, tolmetin, nabumetone, tiaramide, proquazone, bufexamac, flumizole, tinoridine, timegadine, dapsone, diflunisal, benorylate, fosfosal, fenclofenac, etodolac, fentiazac, tilomisole, carprofen, fenbufen, oxaprozin, tiaprofenic acid, pirprofen, feprazone, piroxicam, sudoxicam, isoxicam, celecoxib, Vioxx®, and/or tenoxicam.

### REFERENCES

The following references, to the extent that they provide exemplary procedural or other details supplementary to those set forth herein, are specifically incorporated herein by reference.
U.S. Pat. No. 6,488,953
U.S. Pat. No. 6,737,045
U.S. Pat. No. 6,794,357
U.S. Pat. No. 6,797,258
Akisu et al., Biol. Neonate, 81:260-5, 2002.
Boccia et al., Eur. J. Pediatr., 160:385-391. 2001.
Bury and Tudehope, (Cochrane Review), The Cochrane Library Issue 1, 2001.
Caplan and Jilling, Curr. Opin. Pediatr., 13: 111-115, 2001.
Caplan et al., Gastroenterology, 117:577-83, 1999.
Caplan et al., J. Pediatr. Gastroenterol. Nutr., 24:296-301, 1997.
Carlson et al., Pediatr. Res., 44: 491-98, 1998.
Claud and Walker, FASEB J., 15:1398-1403. 2001.
Dvorák et al., Pediatr. Res., 53:426-33, 2003.
Eibl et al., N. Engl. J. Med., 319:1-7, 1988.
Halac et al., J. Pediatr., 117: 132-8, 1990.
Hentschel et al., Infection, 27:234-238, 1999.
Hoyos, Int. J. Infect. Dis., 3:197-202, 1999.
McGuire and Anthony, Arch. Dis. Child Fetal Neonatal Ed., 88:F11-14, 2003.
Neu, Pediatr. Clin. North Am., 43:409-432, 1996.
Ozturk et al., J. Pediatr. Surg., 37:1330-3, 2002.
Schanler, Pediatr. Clin. North Am., 48:207-219, 2001.
Siu et al., Arch. Dis. Child. Fetal Neonatal Ed., 79:F105-9, 1998.
Zhang et al., J. Amer. Chem. Soc., 125:15288, 2003.

What is claimed is:

1. A method of treating or preventing a condition associated with microbial infection of a subject comprising administering to the subject a composition comprising an effective amount of (a) lysozyme and (b) N-acetyl glucosamine polymer.

2. The method of claim 1, wherein the microbial infection is a viral infection.

3. The method of claim 1 wherein the microbial infection is a respiratory infection, gastrointestinal infection, eye infection, ear infection, nasal infection, or a throat infection.

4. The method of claim 1, wherein the microbial infection is necrotizing enterocolitis.

5. The method of claim 1, wherein the microbial infection is otitis.

6. The method of claim 1, wherein the subject is a newborn.

7. The method of claim 6, wherein the newborn is premature.

8. The method of claim 1, wherein administering is by enteric administration, nasal administration, or topical administration.

9. The method of claim 8, wherein topical administration is by ear drop or eye drop.

10. The method of claim 1, wherein the lysozyme concentration is between 25 mg/L and 800 mg/L.

11. The method of claim 1, wherein the N-acetyl glucosamine polymer concentration is between 5 g/L and 50 g/L.

12. An infant food supplement comprising between 5 g/L and 50 g/L of N-acetyl glucosamine polymer or a mixture thereof, and between 25 mg/L and 800 mg/L of lysozyme.

13. An anti-microbial ear drop composition comprising between 5 g/L and 50 g/L of N-acetyl glucosamine polymer or a mixture thereof, and between 25 mg/L and 800 mg/L of lysozyme.

14. A method of preventing necrotizing enterocolitis comprising supplementing infant formula with a supplement comprising lysozyme and N-acetyl glucosamine polymer.

15. The method of claim 14, wherein the supplement is mixed with an infant formula within one hour of feeding.

16. The method of claim 14, wherein the supplement is fed to an infant prior to feeding with a nutritional formula.

17. The method of claim 14, wherein the supplement is fed to an infant after feeding with a nutritional formula.
